(19) United States
(12) Patent Application Publication
(10) Pub. No.: US 2025/0223099 A1
(43) Pub. Date: Jul. 10, 2025

(54) Incense Aerosol Spray Device and Method of Use

(71) Applicant: Paulette Taylor, Peoria, AZ (US)

(72) Inventor: Paulette Taylor, Peoria, AZ (US)

(21) Appl. No.: 19/007,945

(22) Filed: Jan. 2, 2025

Related U.S. Application Data
(60) Provisional application No. 63/618,558, filed on Jan. 8, 2024.

(51) Int. Cl. B65D 83/141; A61L 9/01; A61L 9/14; A61Q 13/00; B65D 83/18; B65D 83/26

(52) U.S. Cl. CPC B65D 83/141; A61L 9/01; A61L 9/14; B65D 83/18; B65D 83/26; A61Q 13/00

(57) ABSTRACT

An incense aerosol spray device designed to provide incense fragrance without the need for conventional incense burning. The device includes an aerosol can containing an incense composition or mixture. The composition includes essential oils derived from natural sources, solvents for fragrance dispersion, propellants for pressurization, and stabilizers for consistency and stability. The device features a trigger for manual or automatic activation, a pressure-creating chamber for pressurizing the incense mixture, and a nozzle for releasing the aerosol as a fine mist. In one embodiment, the device includes a heating element for melting the composition wherein the composition is in granules or pellets. The device provides a safe and efficient way to disperse fragrance, eliminating the need for open flames or smoke.

### CROSS-REFERENCE TO RELATED APPLICATION

The present application claims priority to, and the benefit of, U.S. Provisional Application No. 63/618,558, which was filed on Jan. 8, 2024, and is incorporated herein by reference in its entirety.

### FIELD OF THE INVENTION

The present invention relates generally to the field of aerosol devices and incense fragrance. More specifically, the present invention relates to a novel incense aerosol spray device. The device is in the form of an aerosol can containing an aerosol composition for releasing incense in aerosol form without burning incense. The device can come in different forms and can be operated manually or automatically. Accordingly, the present disclosure makes specific reference thereto. Nonetheless, it is to be appreciated that aspects of the present invention are also equally applicable to other like applications, devices, and methods of manufacture.

### BACKGROUND

By way of background, incense is a substance that is burned to produce a fragrant scent. Incense is used for counteracting bad or disagreeable odors, repel evil spirits, and/or is used as a vital component in religious practices. Incense is basically an aromatic material and is typically made of plant-based material. Commonly, incense material is burnt to produce fragrance and is made by combining natural fragrant materials such as herbs, resins, essential oils, and wood to create a pleasant aroma when burned.

However, incense burning releases smoke that contains particulate matter, gas products, and other organic compounds. These pollutants can be harmful to human health, particularly when inhaled. Exposure to these pollutants has been linked to airway dysfunction, air pollution, and other health problems. Allergy or asthma can develop at any age, and some individuals have a higher risk of experiencing asthma symptoms when exposed to smoke generated by incense sticks. Burning incense releases irritants such as volatile organic compounds, which can trigger asthma symptoms and exacerbate other respiratory problems.

Moreover, the burning of incense sticks contributes to indoor air pollution, which in turn increases the concentration of carbon monoxide (CO). Carbon monoxide is a colorless, odorless, and tasteless gas that can cause headaches, dizziness, nausea, and other health problems when inhaled in high concentrations. People desire an improved incense that can provide fragrance without requiring to burn incense.

Burning incense can also be a fire hazard. Incense, especially if left unattended, can ignite nearby flammable materials such as curtains, paper, or clothing, potentially causing a fire. The risk of a fire starting increases if the incense is burned too close to these flammable materials or if the incense holder is unstable and prone to tipping over.

Therefore, there exists a long felt need in the art for an improved incense that is not required to burn for providing fragrance. There is also a long felt need in the art for an incense that is in the form of a spray device. Additionally, there is a long felt need in the art for an incense that does not create smoke and pollution. Moreover, there is a long felt need in the art for an aerosol incense spray device. Further, there is a long felt need in the art for an aerosol incense spray device that does not contribute to indoor pollution. Furthermore, there is a long felt need in the art for an incense spray device that is safer and easy to use and is not a fire hazard. Finally, there is a long felt need in the art for an incense aerosol spray device that provides incense in a sprayable aerosol form and overcomes the problems associated with conventional incense.

The subject matter disclosed and claimed herein, in one embodiment thereof, comprises an incense aerosol spray device. The spray device is used for spraying incense fragrance without burning incense and includes an aerosol canister to store an incense composition or mixture, a trigger for manually releasing the stored incense composition in the form of an aerosol spray, a nozzle activated upon triggering of the trigger to release the incense composition as an aerosol, thereby avoiding conventional incense burning. In one embodiment, a heating element is positioned inside the device for heating the composition.

In this manner, the incense aerosol spray device of the present invention accomplishes all of the forgoing objectives and provides users with an improved means for providing incense fragrance which is cleaner, safer, and provides the same spiritual or cultural significance as traditional incense. The incense aerosol spray device, according to the present disclosure, does not create smoke and is easy-to-use and eliminates fire hazard.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an incense aerosol spray device. The spray device comprising an aerosol can is configured to store an incense composition or mixture, the aerosol being cylindrical and suitable for placement in an upright position, a trigger for manually releasing the stored incense composition in the form of an aerosol spray, a nozzle activated upon triggering of the trigger to release the incense composition as an aerosol, thereby avoiding conventional incense burning, a conduit functioning as a means for carrying the composition to a pressure creating chamber, the pressure creating chamber changing the air pressure therein upon pressure exerted by the trigger, causing the pressurized composition to move towards the nozzle, and an electronic actuator configured for automatic actuation of the trigger, the electronic actuator activated using a push button positioned on an external surface of the aerosol can for periodic automatic operation of the device after a pre-determined period of time (i.e., pre-determined time intervals).

In a further embodiment of the present invention, an incense composition for producing fragrance without burning is disclosed. The composition is sprayed using an incense aerosol spray device and the composition comprising one or more essential oils derived from natural sources for imparting a specific fragrance, one or more solvents for dissolving and dispersing the fragrance, one or more propellants for pressurizing the aerosol can and expelling the incense composition when the nozzle is opened, and one or more stabilizers for maintaining the consistency and stability of the incense composition.

In a further embodiment of the present invention, the essential oils form 60%-80% of the composition, the solvents form 10%-15% of the composition, the propellants form 5%-12% of the composition, and the stabilizers form 8%-10% of the composition.

In a further embodiment of the present invention, the composition is in the form of granules and melts using a heating element disposed in the aerosol can.

In a further embodiment of the present invention, a method of spraying incense fragrance using an incense aerosol spray device without burning incense is described. The method comprising the steps of storing an incense mixture inside an aerosol device triggering the aerosol device by a user or automatically triggering the aerosol device using an electronic actuator, pressurizing the incense mixture within the aerosol device using propellants and a pressure creating chamber, thereby creating an aerosol from the incense mixture, spraying the aerosol or mist in the surrounding area using a nozzle, thereby releasing the fragrance without burning the incense material and automatically stopping the spraying of the mist or manually stopping the spraying by pushing the trigger to its original position.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of incense aerosol spray device of the present invention in accordance with the disclosed architecture;

FIG. 2 illustrates a block diagram showing the ingredients of incense composition used in the incense aerosol spray device of the present invention in accordance with the disclosed architecture;

FIG. 3 illustrates a perspective view showing another embodiment of the incense aerosol spray device of the present invention in accordance with the disclosed architecture; and

FIG. 4 illustrates a flow diagram depicting a process of spraying incense in the form of a mist using the incense aerosol spray device in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for an improved incense that is not required to burn for providing fragrance. There is also a long felt need in the art for an incense that is in the form of a spray device. Additionally, there is a long felt need in the art for an incense that does not create smoke and pollution. Moreover, there is a long felt need in the art for an aerosol incense spray device. Further, there is a long felt need in the art for an aerosol incense spray device that does not contribute to indoor pollution. Furthermore, there is a long felt need in the art for an incense spray device that is safer and easy to use and is not a fire hazard. Finally, there is a long felt need in the art for an incense aerosol spray device that provides incense in a sprayable aerosol form and overcomes the problems associated with conventional incense.

The present invention, in one exemplary embodiment, is a method of spraying incense fragrance using an incense aerosol spray device without burning incense. The method comprising the steps of storing an incense mixture inside an aerosol device triggering the aerosol device by a user or automatically triggering the aerosol device using an electronic actuator, pressurizing the incense mixture within the aerosol device using propellants and a pressure creating chamber, thereby creating an aerosol from the incense mixture, spraying the aerosol or mist in the surrounding area using a nozzle, thereby releasing the fragrance without burning the incense material and automatically stopping the spraying of the mist or manually stopping the spraying by pushing the trigger to its original position.

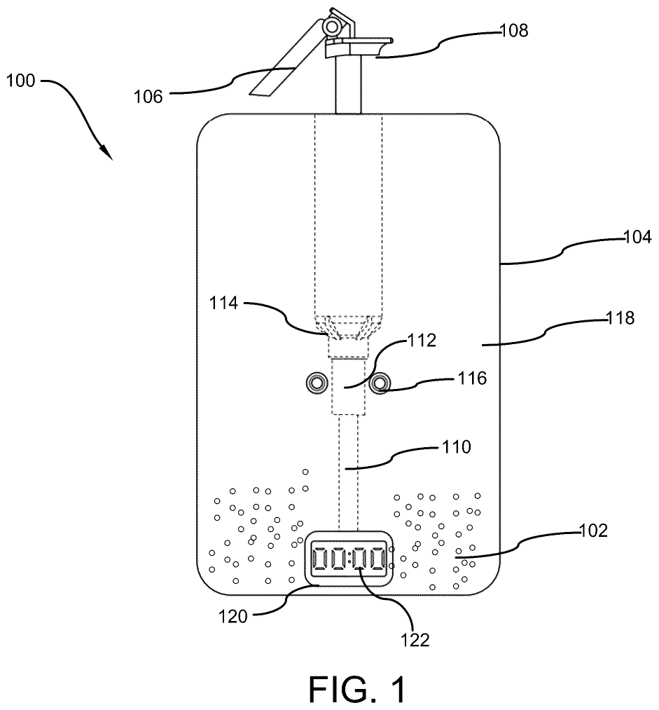

Referring initially to the drawings, FIG. 1 illustrates a perspective view of one potential embodiment of an incense aerosol spray device of the present invention in accordance with the disclosed architecture. The incense aerosol spray device 100 of the present invention is designed to store an incense composition 102 and spray the composition 102 in the form of aerosol for fragrance. More specifically, the aerosol spray device 100 includes an aerosol can 104 which is preferably cylindrical but can be of any geometric shape. The aerosol can 104 is configured to store the incense composition 102 and can be placed upright on any surface. For aerosol spray, the spray device 100 includes a trigger 106 which can be pushed by a user for releasing the stored incense composition 102 in the form of aerosol spray through a nozzle 108. The nozzle 108 is activated upon pressing of the trigger 106 and releases incense composition 102 as mist, thereby preventing conventional burning of incense.

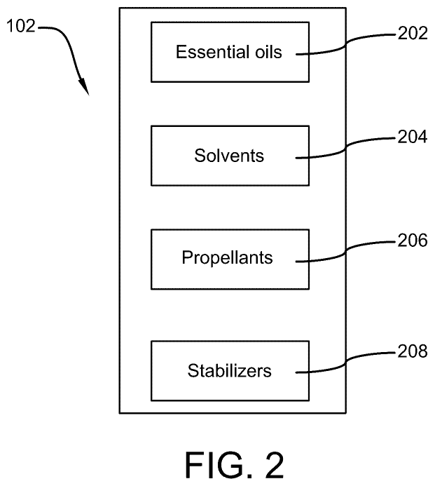

The incense spray device 100 includes a conduit 110 functioning as a means for carrying composition 102 to a pressure creating chamber 112. The pressure creating chamber 112 changes the air pressure therein upon pressure exerted by pressing of the trigger 106 and thus, the pressurized composition 102 moves towards the nozzle 108. It should be noted that the composition 102 includes a propellant as illustrated in FIG. 2 for creating a mist and fine spray.

For automatic operation of the spray device 100, the spray device 100 includes an electronic actuator 114 configured to automatically actuate the trigger 106. The electronic actuator 114 can be activated using a push button 116 positioned on external surface 118 of the aerosol can 104 for automatic operation of the device 100. Then, the electronic actuator 114 automatically actuates the trigger 106 periodically after a pre-determined period of time (i.e., pre-determined time intervals). The pre-determined time period can be pre-configured in the device 100. The aerosol can 104 also includes a timer display 120 which in the preferred embodiment, displays current time 122 in the form of a seven-segment display.

FIG. 2 illustrates a block diagram showing the ingredients of incense composition used in the incense aerosol spray device of the present invention in accordance with the disclosed architecture. The composition 102 includes one or more essential oils 202 for imparting a specific fragrance. The oils 202 can be derived from various natural sources such as flowers, woods, resins, or spices. It should be noted that different combinations and concentrations of oils can be used for creating different scents, such as floral, woody, fruity, herbal aromas and more. In the preferred embodiment, essential oils 202 form 60%-80% of the composition 102 and more preferably, 70%-75% of the composition 102.

The composition 102 includes one or more solvents 204 which are used to dissolve and disperse the fragrance in the incense mixture. The solvents 204 can be alcohol-based or water-based, depending on the type of formulation of the composition 102. The solvents 204 also help to create the aerosol effect when the incense is sprayed from the aerosol device 100. The solvents 204 form 10%-15% of the composition 102.

One or more propellants 206 are included in the composition 102 for pressurizing the aerosol can 104 and expelling the incense composition 102 when the nozzle 108 is opened. The propellant 206 can be in gaseous or liquid form, such as compressed air, butane, or propane. The propellant 206 together with the pressure creating chamber 112 creates the excessive pressure and force to transform the incense mixture 102 into an aerosol spray. The propellants 206 form 5%-12% of the composition 102.

One or more stabilizers 208 are used in the composition 102 for maintaining the consistency and stability of the incense composition 102. The stabilizers 208 are used for mixing other ingredients and preventing separation thereof over time. The stabilizers 208 promote the formation of stable emulsions and prevent the separation of oil and water phases of the incense composition 102. The stabilizers 208 can include one or more polysorbates, lecithin, glyceryl stearate, and more. Stabilizers 208 can also include antioxidants which prevent the oxidation or degradation of the fragrance oils 202. The stabilizers form 8%-10% of the composition 102.

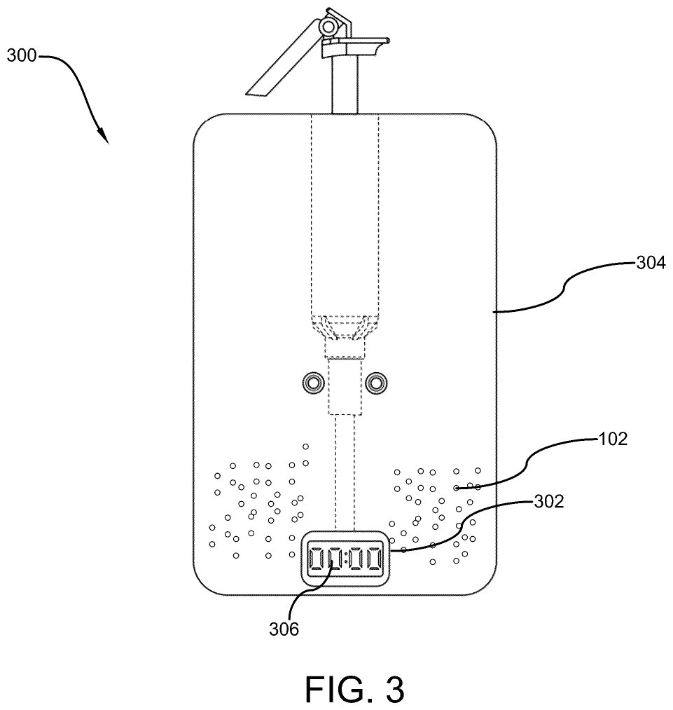

FIG. 3 illustrates a perspective view showing another embodiment of the incense aerosol spray device of the present invention in accordance with the disclosed architecture. In the present embodiment, the incense spray device 300 has a similar spraying structure like device 100 of FIG. 1. Further, the device 300 includes a heating element 302 positioned inside the aerosol can 304 for heating the incense mixture 102. The heating element 302 is designed to reach a specific temperature, preferably in the range of 10 degrees centigrade to 20 degrees centigrade to heat the incense mixture 102 without causing combustion. Furthermore, the incense mixture 102 in the present embodiment can be in the form of pellets or granules to gradually release fragrance upon melting or vaporizing due to the heating element 302. The heating element 302 can include a built-in time module 306 for preconfigured deactivation to stop producing incense aerosol spray.

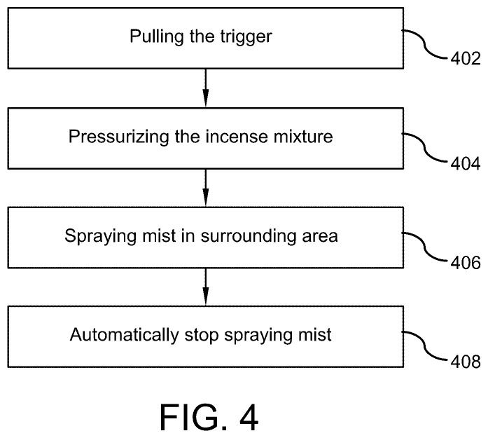

FIG. 4 illustrates a flow diagram depicting a process of spraying incense in the form of a mist using the incense aerosol spray device in accordance with the disclosed architecture. Initially, the trigger of the aerosol device is pressed by a user (Step 402). Alternatively, the trigger can be automatically triggered using the electronic actuator as described in FIG. 1. Then, using the propellants and the pressure creating chamber 112, the incense mixture is pressurized to create an aerosol from the incense mixture (Step 404). Using the nozzle, the aerosol or mist is sprayed in the surrounding area to create a fragrance without burning the incense material (Step 406). Finally, the spraying of the mist is automatically stopped or can be manually stopped by pushing the trigger to the original position thereof (Step 408).

The aerosol device 100 of the present invention is reusable and can come in different sizes such as compact-sized devices for travel or on-the-go use. The device 100 can also be designed for everyday use and offers a balance between portability and a sufficient amount of incense.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “incense aerosol spray device”, “incense spray device”, “spray device”, “spray device”, and “device” are interchangeable and refer to the incense aerosol spray device 100,300 of the present invention.

Notwithstanding the forgoing, the incense aerosol spray device 100,300 of the present invention can be of any suitable size and configuration as is known in the art without affecting the overall concept of the invention, provided that it accomplishes the above stated objectives. One of ordinary skill in the art will appreciate that the incense aerosol spray device 100,300 as shown in the FIGS. are for illustrative purposes only, and that many other sizes and shapes of the incense aerosol spray device 100,300 are well within the scope of the present disclosure. Although the dimensions of the incense aerosol spray device 100,300 are important design parameters for user convenience, the incense aerosol spray device 100,300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An incense aerosol spray device comprising:
an incense aerosol composition housed in an aerosol can;
wherein said aerosol can having a trigger, a nozzle, a conduit, and a pressure creating chamber;
wherein said trigger actuates said nozzle upon pressing of said trigger;
wherein upon said actuation of said nozzle said incense aerosol composition is carried through said conduit to said pressure creating chamber and out said nozzle;
wherein said pressure creating chamber changes the air pressure therein upon pressure exerted by pressing of said trigger;
wherein said incense aerosol composition is released as a mist from said nozzle; and
further wherein said incense aerosol composition having a plurality of essential oils, a solvent, a propellant, and a stabilizer.

2. The incense aerosol spray device of claim 1, wherein said plurality of essential oils selected from a group consisting of a flower oil, a wood oil, a resin, and a spice oil.

3. The incense aerosol spray device of claim 2, wherein said plurality of essential oils form 60%-80% of the volume of said incense aerosol composition.

4. The incense aerosol spray device of claim 3, wherein said plurality of essential oils form 70%-75% of the volume of said incense aerosol composition.

5. The incense aerosol spray device of claim 4, wherein said solvent is alcohol-based.

6. The incense aerosol spray device of claim 4, wherein said solvent is water-based.

7. The incense aerosol spray device of claim 5, wherein said solvent forms 10%-15% of the volume of said incense aerosol composition.

8. The incense aerosol spray device of claim 7, wherein said propellant is selected from a group consisting of a compressed air, a butane, and a propane.

9. The incense aerosol spray device of claim 8, wherein said propellant forms 5%-12% of the volume of said incense aerosol composition.

10. The incense aerosol spray device of claim 9, wherein said stabilizer is selected from a group consisting of a polysorbates, a lecithin, a glyceryl a stearate, and an antioxidant.

11. The incense aerosol spray device of claim 10, wherein said stabilizer forms 8%-10% of the volume of said incense aerosol composition.

12. An incense aerosol spray device comprising:
an incense aerosol composition housed in an aerosol can;
wherein said aerosol can having a trigger, a nozzle, a conduit, a pressure creating chamber, and an electronic actuator;
wherein said trigger actuates said nozzle upon pressing of said trigger;
wherein upon said actuation of said nozzle said incense aerosol composition is carried through said conduit to said pressure creating chamber and out said nozzle;
wherein said electronic actuator automatically actuates said trigger;
wherein said electronic actuator having a push button on an external surface of said aerosol can for automatic operation of said incense aerosol spray device;
wherein the automatic operation including said electronic actuator automatically actuating said trigger at pre-determined time intervals;
wherein said pressure creating chamber changes the air pressure therein upon pressure exerted by pressing of said trigger;
wherein said incense aerosol composition is released as a mist from said nozzle; and
further wherein said incense aerosol composition having a plurality of essential oils, a solvent, a propellant, and a stabilizer.

13. The incense aerosol spray device of claim 12, wherein said plurality of essential oils selected from a group consisting of a flower oil, a wood oil, a resin, and a spice oil.

14. The incense aerosol spray device of claim 13, wherein said plurality of essential oils form 70%-75% of the volume of said incense aerosol composition.

15. The incense aerosol spray device of claim 14, wherein said solvent forms 10%-15% of the volume of said incense aerosol composition.

16. The incense aerosol spray device of claim 15, wherein said propellant is selected from a group consisting of a compressed air, a butane, and a propane.

17. The incense aerosol spray device of claim 16, wherein said propellant forms 5%-12% of the volume of said incense aerosol composition.

18. The incense aerosol spray device of claim 17, wherein said stabilizer is selected from a group consisting of a polysorbates, a lecithin, a glyceryl a stearate, and an antioxidant.

19. The incense aerosol spray device of claim 18, wherein said stabilizer forms 8%-10% of the volume of said incense aerosol composition.

20. An incense aerosol spray device comprising:
an incense aerosol composition housed in an aerosol can;
wherein said aerosol can having a trigger, a nozzle, a conduit, a pressure creating chamber, and a heating element;
wherein said trigger actuates said nozzle upon pressing of said trigger;
wherein upon said actuation of said nozzle said incense aerosol composition is carried through said conduit to said pressure creating chamber and out said nozzle;
wherein said pressure creating chamber changes the air pressure therein upon pressure exerted by pressing of said trigger;
wherein said heating element heats said incense aerosol composition to a temperature from 10 degrees centigrade to 20 degrees centigrade;
wherein said incense aerosol composition is released as a mist from said nozzle;
wherein said incense aerosol composition having a plurality of essential oils, a solvent, a propellant, and a stabilizer;
wherein said plurality of essential oils selected from a group consisting of a flower oil, a wood oil, a resin, and a spice oil;
wherein said plurality of essential oils form 70%-75% of the volume of said incense aerosol composition;
wherein said solvent forms 10%-15% of the volume of said incense aerosol composition;
wherein said propellant is selected from a group consisting of a compressed air, a butane, and a propane;
wherein said propellant forms 5%-12% of the volume of said incense aerosol composition;
wherein said stabilizer is selected from a group consisting of a polysorbates, a lecithin, a glyceryl a stearate, and an antioxidant; and
further wherein said stabilizer forms 8%-10% of the volume of said incense aerosol composition.
